(19) United States
(12) Patent Application Publication
(10) Pub. No.: US 2014/0046250 A1
(43) Pub. Date: Feb. 13, 2014

(54) STEERABLE ELEMENT FOR USE IN SURGERY

(71) Applicants: Ajay Kumar Jain, Islington (GB); Andrew Douglas McCulloch, Ipswich (GB)

(72) Inventors: Ajay Kumar Jain, Islington (GB); Andrew Douglas McCulloch, Ipswich (GB)

(73) Assignee: Barts and the London NHS Trust, London, EN (GB)

(21) Appl. No.: 14/001,647

(22) PCT Filed: Mar. 14, 2012
(86) PCT No.: PCT/GB2012/050555

§ 371 Date: Oct. 29, 2013

(30) Foreign Application Priority Data
Mar. 15, 2011 (GB) 1104384.1

(51) Int. Cl. A61M 25/01

(52) U.S. Cl. CPC A61M 25/0155
USPC 604/95.03

(57) ABSTRACT

A steerable element for use in surgery, comprising: an inflatable member; and an elongate frame azimuthally surrounding said inflatable member, wherein: said inflatable member is configured to press against said elongate frame on inflation so as to cause a change in the curvature of said elongate frame. A catheter, an insertion system, a medical implant comprising the steerable element, a delivery system comprising the steerable element, and a method of configuring the steerable element for use.

The present invention relates to a steerable element for use surgery, particularly in a minimally invasive procedure, for example as part of a catheter or a medical implant.

Current interventional techniques require access to internal cavities of the body via natural orifices, for example via oral, nasal, rectal or vaginal routes or via percutaneous routes, for example vascular, gastrointestinal, bone, kidney and cardiac access.

In order to gain access to these internal sites a device is required which connects the site of interest with the clinician, over or through which an implant can be delivered or a therapeutic action imparted. The route to these internal sites can be tortuous and convoluted, making access difficult and increasing the risk of injury.

For example, for vascular access to the heart, the route from the femoral artery (a typical access point) to the cardiac tissue will have to traverse the aortic arch which turns through over 180 degrees to reach the aortic valve and coronary vessels.

One key issue with guiding a delivery device through a blood vessel is the risk of touching the blood vessel wall and knocking off calcified deposits or thrombus (blood clot) causing emboli. This problem is most acute in the aortic arch, where emboli can travel up the head and neck vessels, causing a stroke. The contact of large and/or stiff devices may also cause vascular trauma.

Catheters or tubes are known for delivery of large cardiac implants such as percutaneous heart valves which are designed to try to minimise constact with walls of the aortic arch. However, they have several shortcomings.

Once such arrangement is described in U.S. Pat. No. 7,780,723 (Edwards Lifesciences). Here, a catheter system is disclosed that has a steerable catheter characterised by a pull-wire arrangement which biases the catheter in a particular direction. This method of steering requires the whole catheter to be stiff enough to provide a reaction force against the pulling of the wire.

Pull-wire steering is also used in catheters for other applications where accurate positioning is required. U.S. Pat. No. 5,882,346 and U.S. Pat. No. 7,717,899 disclose use of wire control systems for electrophysiology mapping and ablation purposes in the heart. These devices still require a stiff proximal section to the catheter in order to impart the curvature via pulling of the wire.

Another known method of catheter deflection is to use a balloon either asymmetrically positioned on the outside of the device or a curved balloon. The major advantage of this method over pull-wire devices is that the proximal catheter section does not need to be stiff to react against pulling forces. WO2010/078112 describes an arrangement of balloons, which when inflated symmetrically, can cause the catheter to curve. US493275 has a precurved balloon which causes a curvature when inflated.

The major drawback with asymmetrical balloons is the inherent lack of stiffness required in the catheter to allow for bending. This can create pushability problems for cardiac catheters delivering bulky payloads such as heart valves. Precurved balloons also require relatively soft catheters to allow bending; also the degree of curvature is limited by the precurved shape of the balloon.

It is an object of the invention to address at least some of the problems with the prior art discussed above.

According to an aspect of the invention, there is provided a steerable element for use in surgery, comprising: an inflatable member; and an elongate frame azimuthally surrounding said inflatable member, wherein: said inflatable member is configured to press against said elongate frame on inflation so as to cause a change in the curvature of said elongate frame.

Surgery in this context is intended to cover any intervention in the body where navigation/access is needed, such as laparoscopy, natural orifice surgery or endoscopy. The term surgery thus encompasses so-called minimally invasive procedures, and, optionally, other procedures.

When applied to a delivery device, the use of steerable element according to the above aspect of the invention obviates the need for stiffness in a proximal end of a delivery device (e.g. catheter) associated with the steerable member, as is the case with the pull-wire arrangements discussed above, because bending of the element is achieved entirely by inflation of the inflatable member. Additionally, the portion of the delivery device that is adjacent to the steerable element does not have to be made as soft as in embodiments that rely on an asymmetrically positioned balloon.

More generally, arranging for the inflatable element to interact with an elongate frame that surrounds the inflatable element azimuthally, rather than simply to press against the frame from one side, allows considerably more flexibility and control in terms of how the element as a whole deforms in response to inflation of the inflatable member. In the prior art, the only possibility is general bending to one side, which is not accurately controllable either in shape or direction. In addition, in contrast to the use of a balloon to one side of the catheter tip, the present embodiment allows the catheter tip to be selectively stiffened to assist with insertion into the patient and then subsequently softened for advancement to the site of interest with a minimum of damage to tissue.

Optionally, the elongate frame comprises a continuous spine that extends longitudinally. This provides the steerable element with longitudinal compressive strength and/or greater resistance to buckling, which facilitate pushing of the steering element along vessels within the patient while still allowing the necessary lateral bending associated with actuation of the steerable element.

Optionally, the elongate frame comprises a plurality of spines that are longitudinally and/or azimuthally displaced relative to each other, so that the steerable element can adopt complex shapes on actuation. Optionally, the elongate frame is tailored according to imaging data representing the relevant anatomy of the patient, so as to conform advantageously to the anatomy where the steerable element is to be deployed, for example in a region of tortuous anatomy at an intermediate position between the proximal and distal ends of a catheter or at a treatment site at the distal end of a catheter, or in a medical implantOptionally, the steerable element is incorporated into a delivery device. A delivery device in this context is any catheter based device that is configured to be used as a means of delivering an implant, medical therapy, energy or the like from outside the body to the site of interest.

Optionally, the steerable element is incorporated into the catheter itself, or into a medical implant.

Optionally, the steerable element is configured to curve progressively from one end to the other. For example, the steerable element may be made to curve more quickly at the tip of the steerable element than at the base of the steerable element. In this situation the pressure within the inflatable member may be made to increase progressively as the steerable element is pushed round a sharp corner, such that the curve of the steerable element advances down the steerable element at the same time as the steerable element moves round the corner. The steerable element can thus be “steered” round the corner with a minimum of stress being imparted to walls of the vessel.

Embodiments of the invention will now be described, by way of example only, with reference to the accompanying schematic drawings in which corresponding reference symbols indicate corresponding parts, and in which:

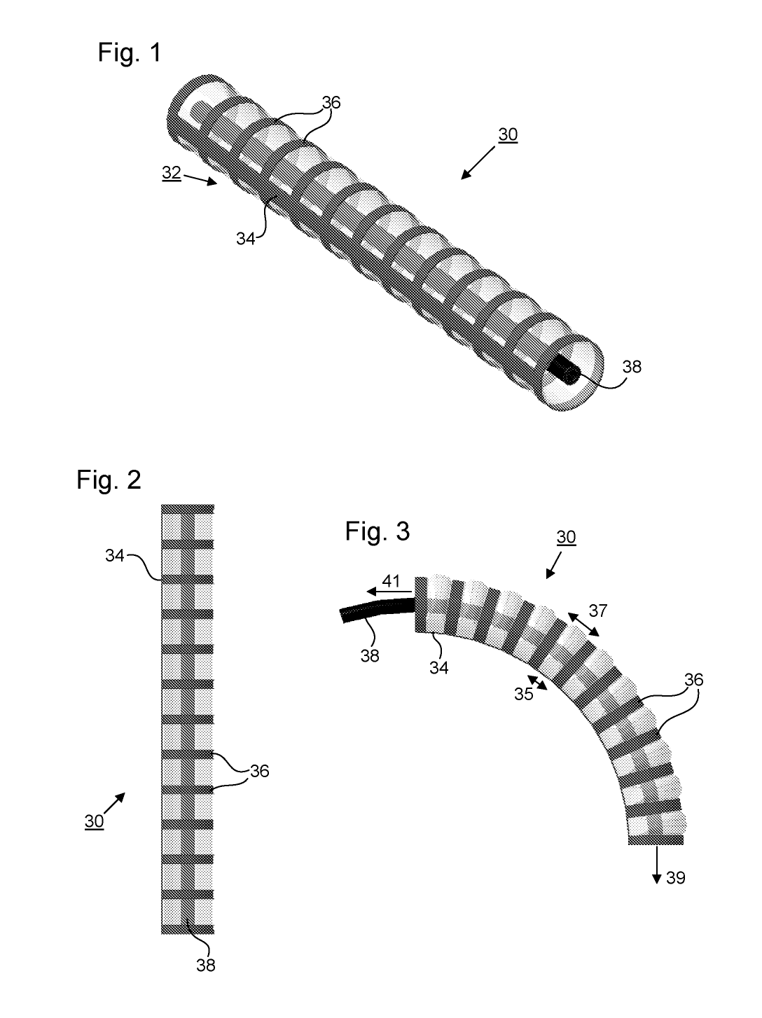

FIG. 1 is a schematic perspective view of an unactuated steerable member according to an embodiment of the invention;

FIG. 2 is a schematic side view of the unactuated steerable member of FIG. 1;

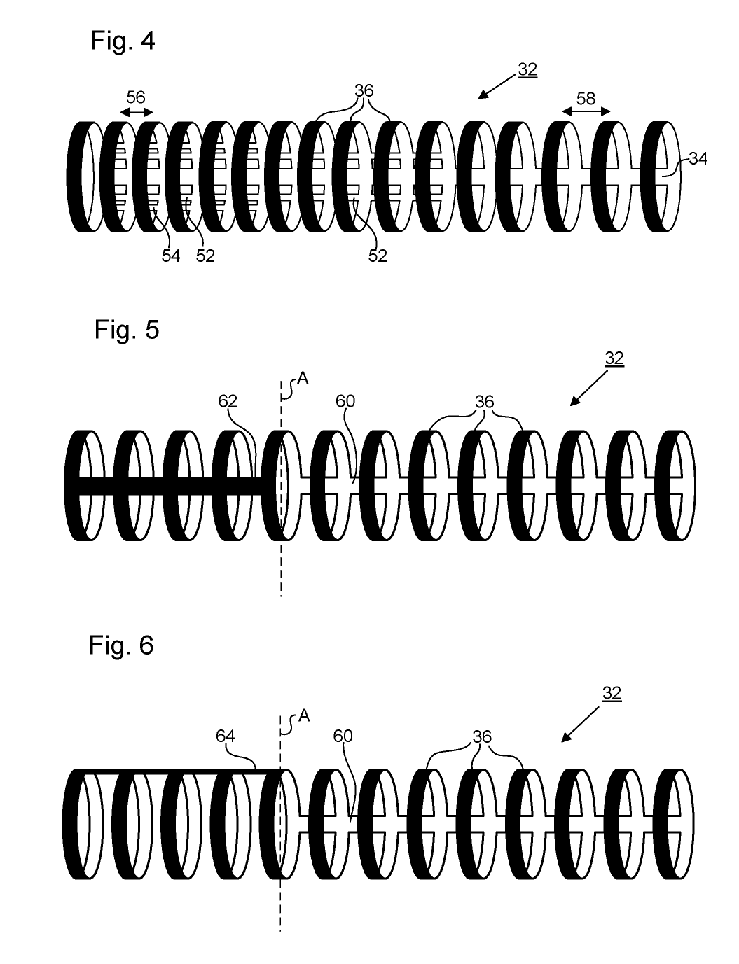

FIG. 3 is a schematic side view of the steerable member of FIG. 1 in an actuated state;

FIG. 4 is a schematic illustration of an elongate frame having a resistance against inflation of the inflatable member that increases progressively along its length;

FIG. 5 is a schematic illustration of an elongate frame having two longitudinally displaced spines, offset azimuthally relative to each other by 180 degrees;

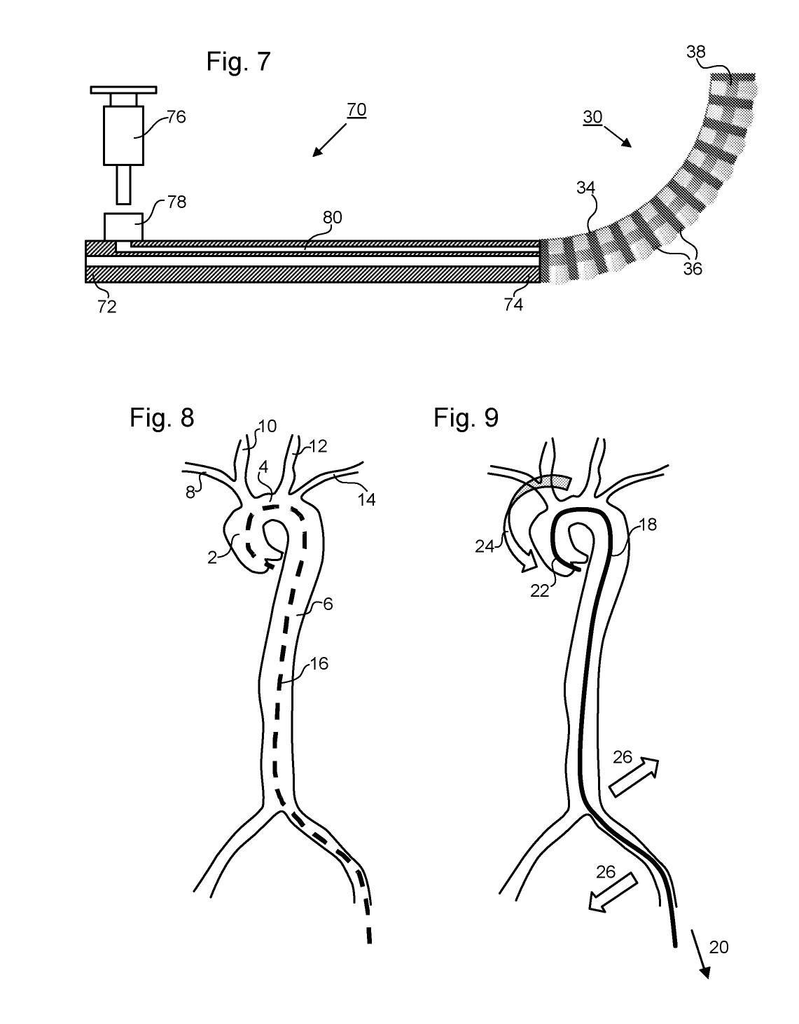

FIG. 6 is a schematic illustration of alternative elongate frame, of the general type shown in FIG. 7 except that the two spines are offset aximuthally relative to each other by 90 degrees rather than 180 degrees;

FIG. 7 is a schematic side view of the steerable member of FIGS. 3 to 5 as part of a catheter and positioned at the distal end thereof;

FIG. 8 is schematic illustration of a required trajectory for a catheter to access the aortic valve or coronary vessels;

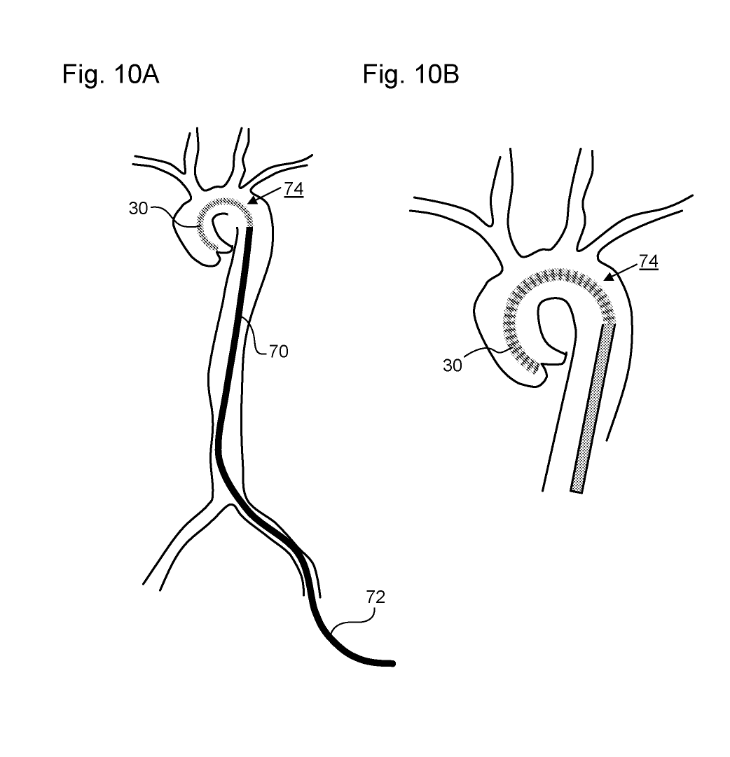

FIG. 9 is a schematic illustration of a prior art approach to steering a catheter along the trajectory of FIG. 8;

FIGS. 10A and 10B illustrate use of a steerable element to assist with navigating the trajectory of FIG. 8; FIG. 10A shows the whole trajectory; FIG. 10B is larger view of the region at the distal end of the catheter;

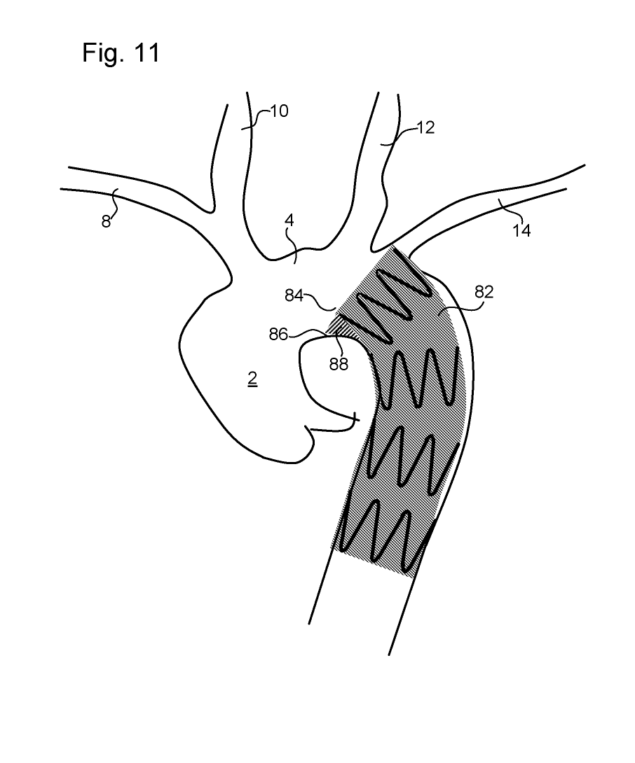

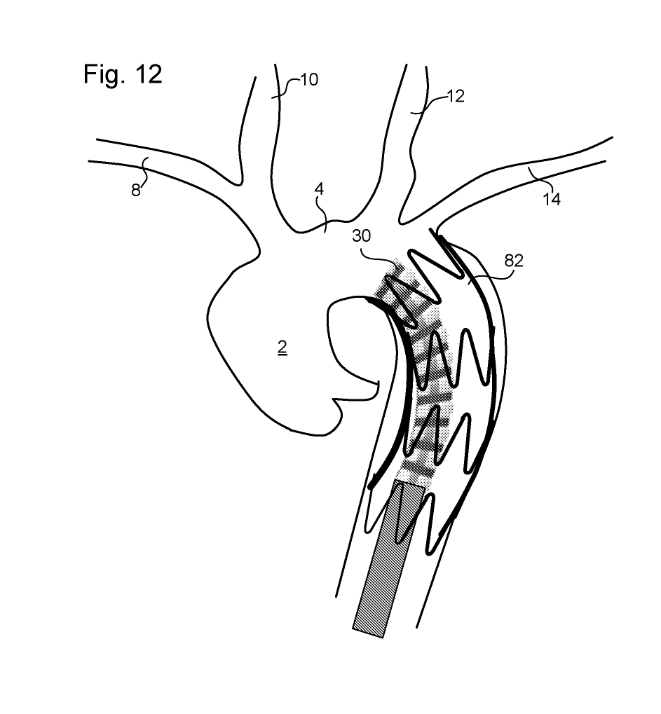

FIG. 11 is a schematic illustration of a thoracic aortic stent graft positioned just distally to the head and neck vessel of the aortic arch, with a leading edge of the stent graft sitting free from the aortic surface due to excessive stiffness of the stent graft;

FIG. 12 is schematic illustration of use of a steering element according to a disclosed embodiment to maneouvre and/or expand the stent graft of FIG. 8 into a more effective position;

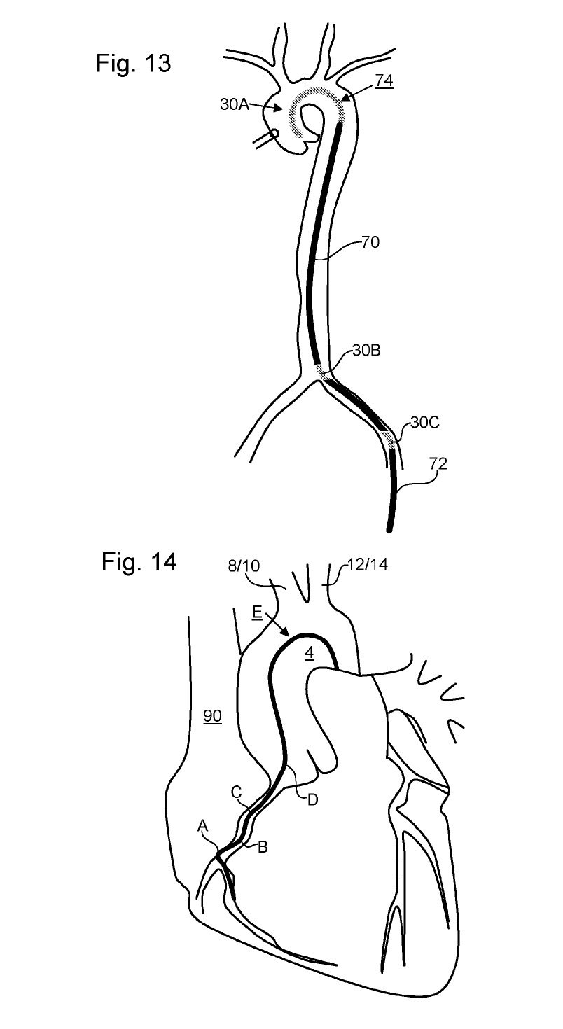

FIG. 13 is a schematic illustration of a bespoke catheter system using a plurality of steerable elements at different points along the length of the catheter;

FIG. 14 is a schematic illustration of a further bespoke catheter system for navigating effectively through tortuous coronary blood vessels, for example to form a resilient delivery system;

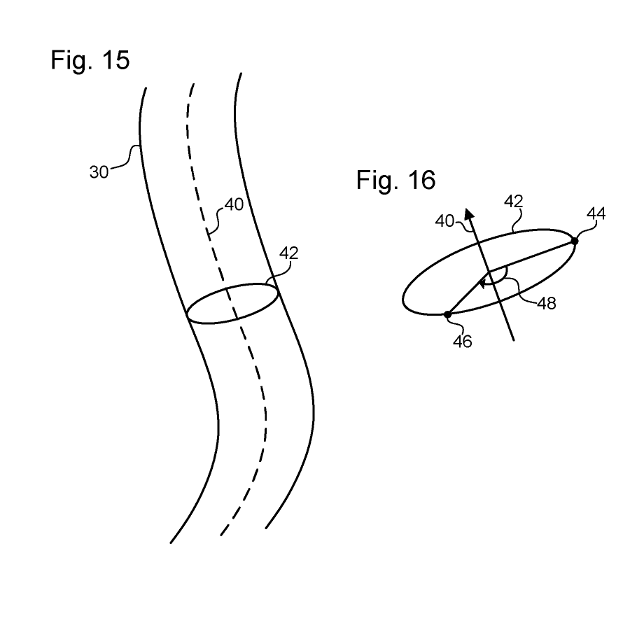

FIG. 15 is a schematic illustration of a longitudinal axis of the elongate frame; and

FIG. 16 is a schematic illustration of azimuthally separated points in a cross-section of the elongate frame.

FIGS. 1 and 2 show an example of a steerable element 30 for use in surgery, in an unactuated state, in perspective and side views respectively. In this example, the element 30 is straight in the unactuated state, but other configurations are possible, including single-curved (i.e. curvature describable by reference to a single axis and in a single sense, right-handed or left-handed, relative to the axis) or multiple-curved geometries (i.e. curvatures describable by reference to a plurality of axes and/or senses of curvature).

The element 30 comprises an elongate frame 32 having a spine 34 and a plurality of rings 36 azimuthally surrounding a longitudinal axis of the element 30. A flexible core lumen 38 runs along the longitudinal axis for accommodating devices to be fed through the steerable element 30, for example a guidewire or guidewires, which can be used if necessary to provide further measures to aid with deployment and positioning.

The longitudinal axis is an axis that extends parallel to the elongation of the elongate frame 32, at least roughly along a cross-sectionally central line thereof. Where the elongate frame 32 is curved the longitudinal axis will also be curved. In the simplest embodiment, the elongate frame 32 will take a locally cylindrical form (i.e. a form having cylindrical symmetry, optionally in the form of a cylinder with a substantially circular cross-section of constant radius over at least a short length of the elongate frame; over longer distances, the radius of the cross-section may vary and the axis may also deviate from a straight line to follow any longitudinal curvature of the elongate frame 32.

FIGS. 15 and 16 show an example geometry to illustrate the intended meaning of the term “longitudinal axis” and similar, and “azimuthally surrounding” and similar. Here, the outer geometry of the steerable element 30 takes a curving, substantially cylindrical form with a correspondingly curved longitudinal axis 40 running along the centre thereof. A sample cross-section 42 is shown and takes a substantially circular form. FIG. 16 is an enlarged view of the sample cross-section 42, with two sample points 44 and 46 both lying in the plane of the cross-section 42 (i.e. the plane perpendular to the longitudinal axis 40 where it crosses the plane) but azimuthally separated from each other by azimuthal angle 48.

Referring once again to FIGS. 1 and 2, the elongate frame 32 is configured such that when an inflatable member is inflated (expanded) within the elongate frame 32, so as to push outwards against the inner surface of the elongate frame 32 (in the example shown, this would be against the inner surfaces of the plurality of rings 36 and of the spine 34), the elongate frame 32 deforms in an azimuthally asymmetric manner. In the case where the elongate frame 32 is straight in the unactuated state (i.e. before the inflatable member is inflated), this will cause the elongate frame 32 to bend.

The azimuthally asymmetric deformation can be made to occur by arranging for the elongate frame to resist longitudinal expansion in an azimuthally asymmetric manner. In the example shown, this is achieved by providing the elongate frame 32 with a spine 34 that extends continuously along a direction parallel to the longitudinal axis of the elongate frame 32 and a plurality of rings rings 36 that azimuthally surround the longitudinal axis (and hence the inflatable member) and which are each connected to the spine 34 at a single point. When the inflatable member is inflated, longitudinal relative movement of the rings 36 is restricted more at the spine 34 then at other points around the rings 36. In particular, relative longitudinal movement of the rings 36 is considerably less restricted on the side of the longitudinal axis opposite to the spine 34 than it is at the spine 34. The result is that on inflation of the inflatable member the rings 36 will tend to be forced further apart on this side than at the spine, which results in a bending of the element 30 about an axis on the spine side of the longitudinal axis.

FIG. 3 is a schematic illustration of the actuated state of a steerable element 30 of the type illustrated in FIGS. 1 and 2. As can be seen, the separation 35 between adjacent rings 36 at the spine is smaller than the separation 37 between adjacent rings on the opposite side, due to azimuthally asymmetric deformation of the elongate member 32 in response to inflation of the inflatable member.

The deformation that results from actuation of the steerable element 30 may be made to take a variety of forms by varying the configuration of the elongate frame.

In the example shown in FIG. 3, the elongate frame 30 is configured to provide a curvature that does not change as a function of longitudinal position along the element 30. The curvature is thus fully defined by reference to a single radius of curvature about a single axis. As the degree of inflation of the inflatable member is increased, the radius of curvature will decrease, reflecting the increasing curvature. Similarly, the relative angle between the longitudinal axis 39 at one end of the element 30 relative to the longitudinal axis 41 at the other end, which may be seen as a measure of the amount of “steering” that has been imparted, will also increase by a corresponding amount. In the example shown, the relative angle is about 90 degrees.

The degree of inflation is controlled by controlling the pressure within the inflatable member. As this can be achieved accurately over a continuous range of pressures using standard methods in the art, it is possible to control the degree of distortion of the steerable element 30 with corresponding accuracy and over a correspondingly continuous range. This is in contrast to prior art arrangements, such as the pull-wire systems, where it is difficult or impossible to vary the degree of actuation over a continuous range with satisfactory accuracy. Indeed, for practical purposes these systems are essential binary with respect to actuation and, as a result, can be used less flexibly than embodiments of the present invention.

More complex distortions can be obtained by arranging for a variation with longitudinal position of one or both of the following: 1) the strength of the elongate frame, in particular the resistance of the elongate frame to longitudinal extension; and 2) the nature of the azimuthal asymmetry.

FIG. 4 shows an example of an elongate frame 32 having a resistance to longitudinal extension that varies according to variation (1). Here, additional longitudinal reinforcing members 52, azimuthally displaced from the centre of the spine 34, are introduced in a middle section of the elongate frame 32, and further additional reinforcing members 54 are provided in a proximal (left) section of the elongate frame 32. This alone would cause the response of the elongate frame (e.g. the degree of curvature) to be progressively lower moving from the distal end (right) to the proximal end (left) of the elongate frame 32. However, in the present example, the relative spacing between the rings 36 is also varied, which will tend further to cause the response of the elongate frame to be progressively lower moving from the distal end to the proximal end. This sort of response may also be described in terms of a radius of curvature that decreases continuously as a function of position from one end of the elongate frame to the other for a given degree of inflation. Other functions of radius of curvature are also possible. For example, it may be arranged for the maximum or minimum radius of curvature to occur at an intermediate longitudinal position.

The variation of relative spacing of the rings 36 and the inclusion of the additional reinforcing members 52 and 54 are provided in the same elongate member 32 in this example, but they could each be provided separately without departing from the scope of the invention. The variation in curvature could also be reversed with respect to the proximal and distal ends. Similarly, other ways of implementing variation (1) are possible. For example, the thickness or material of the elongate frame 32 could be varied as a function of longitudinal position.

FIGS. 5 and 6 show examples of elongate frames where the nature of the azimuthal asymmetry varies as a function of longitudinal position (variation (2)). In both examples, this is achieved by providing two spines (first and second spines 60 and 62 in FIG. 5, and first and scond spines 60 and 64 in FIG. 6). In FIG. 5, the first spine 60 is provided to the right of position A, and the second spine 62, displaced azimuthally by 180 degrees is positioned to the left of position A. This arrangement results in a difference in the sense of curvature induced to the elongate frame 32 on actuation on either side of position A. Referring to the page of the Figure, to the right hand side of position A, inflation will cause a curvature around an axis that is below the plane of the page, while the curvature to the left hand side of position A will be around an axis that is above the plane of the page. If this elongate frame 32 is considered as “pointing” to the left in the Figure, such curvature may be considered to be “right-handed” to the right of position A and “left-handed” to the left of position A.

In FIG. 6, the first spine 60 is provided to the right of position A in the same azimuthal position as the first spine 60 in FIG. 5. In contrast to the arrangement of FIG. 5, however, the second spine 64 of FIG. 6 is azimuthally displaced by 90 degrees relative to the first spine 60. This arrangement causes a differences in the axis about which curvature is induced on actuation either side of position A. Referring to the page of the Figure, to the right hand side of position A, inflation will cause a curvature about an axis that is horizontal and below the plane of the page, while the curvature to the left hand side of position A will be around an axis that is perpendicular to the plane of the page and above the position of the elongate frame 32 on the page.

The two examples in FIGS. 5 and 6 involve a single discrete change in the symmetry of the elongate frame 32 (at position A). However, other arrangements are possible. For example, more than two spines may be provided. Alternatively or additionally, a spine may be provided that is capable of providing a continuously varying azimuthal symmetry. This may be achieved by orienting the spine in a direction other than parallel to the longitudinal axis of the elongate frame 32. For example, a spine may be provided that spirals around a part of the elongate frame, which will tend to cause a corresponding spiral deformation of the elongate frame on actuation.

In fact, the approach of the present invention provides the possibility of tailoring the shape of the steerable element in a highly flexible manner, both by varying properties of the elongate frame and, in use, by varying the pressure in the inflatable member.

FIG. 7 illustrates how a steerable element 30 may be used as part of a catheter 70. The catheter 70 comprises a proximal end 72, which would normally remain outside of the human or animal body to be treated by the catheter 70, and distal end 74, which is fed into the body and driven to the site of interest. The relative proportions of the catheter 70 have been altered for clarity; in reality the ratio of the distance between the proximal and distal ends would be much greater. In the present embodiment, the steerable element 30 is located at the distal end 74. The steerable element 30 is particularly useful at the distal end 74: firstly, because the distal end 74 is inserted into the patient first, so there are advantages to be had in being able to control the stiffness of the distal end 74 to aid in this process, for example by partially or completely inflating the inflatable member; and, secondly, because it is the distal end 74 that is the leading end throughout the insertion process, so that there are benefits to be had in being able to achieve low stiffness (by deflating the inflatable element) while the distal end is en route to the site of interest, before inflating the inflatable member in order to steer the distal end into the desired position at the site of interest (and/or, indeed, to help steer the distal end 74 through tortuous sections of anatomy en route). However, the steerable element 30 may alternatively or additionally be positioned at an intermediate point along the catheter. For example, the steerable element 30 may be located at a point on the catheter that will correspond to a tortuous section of the vessel through which the catheter is fed when the distal end has reached the site of interest. In this way, it is possible to reduce the stresses imparted on the vessel by the present of the catheter during treatment. This approach may be particularly practical where the catheter is tailor made for an individual patient.

In the example shown, the catheter 70 has a flexible internal lumen 38 running continously from the proximal end 72 to the tip of the steerable element 30 at the distal end. A pressure control system is provided for controlling the pressure in the inflatable member within the steerable element 30. In the example shown, the pressure control system 76 comprises a hand-operated syringe 76 configured to couple with a valve 78, and pressure fluid lumen 80. However, other arrangements are possible. For example, an active control system may be provided to control the pressure, comprising means for measuring the pressure in the pressure fluid lumen 78 and/or in the inflatable member, and adjusting the pressure to achieve a target setpoint pressure, for example using a feedback circuit. A powered bellows or piston system may be used to increase or decrease the pressure, for example.

Various example situations are now described in which one or more steerable elements of disclosed embodiments may be used.

FIG. 8 shows a typical anatomical arrangement of the aorta (comprising descending aorta 2, aortic arch 4, and according aorta 6), together with head and neck vessels (comprising right subclavian artery 8, right common carotid artery 10, left common carotid artery 12, and left subclavian artery 14). Broken line 16 shows the general trajectory that a catheter would need to follow during an interventional procedure to reach the aortic valve or coronary vessels.

FIG. 9 illustrates the result of a prior art approach to achieving such a trajectory using a pull-wire catheter 18. As the wire (not shown) is pulled back in the direction of arrow 20, the catheter tip 22 curves (curved arrow 24). However, this action results in compressive forces being applied to the catheter 18 that tend to cause it to straighten and align with the tensioned pull wire. This causes unwanted forces to be applied to the tortuous anatomy of the aorta (arrows 26) increasing the risk of damage to these delicate tissues.

This problem can be addressed by incorporating one or more steerable elements 30 according to embodiments disclosed herein into the catheter instead of the pull-wire arrangement, because the steerable elements 30 are actuatable without inducing additional tension into the catheter. FIGS. 10A and 10B illustrate one way in which this may be achieved, using a steerable element 30 of the type illustrated in FIGS. 1 to 3. FIG. 10A shows the whole route of the catheter from proximal end 72, outside of the body to be treated, to the distal end 74 at the site to be treated. FIG. 10B is an enlarged view of the site to be treated, showing the actuated steerable element 30 in further detail. The resulting position of the distal end 74 of the catheter 70 is similar to that of the pull-wire prior art catheter 18 shown in FIG. 9, but the proximal end 72 of the catheter 70 is not affected by the inflation and subsequent curving at the tip, and unwanted forces applied to delicate tissues in tortuous anatomy regions near the proximal end 72 are greatly reduced.

FIG. 11 shows a thoracic aortic stent graft 82 placed just distally of the head and neck vessels 8/10/12/14 of the aortic arch 4. As the graft 82 is relatively stiff the curve of the aortic arch 4 has caused the leading edge 84 of the graft 82 to sit free from the aortic surface 86, leaving a gap (hatched area 88). This “malapposition” is undesirable and can lead to thrombus formation, stroke, stent graft migration and graft failure.

FIG. 12 shows how a steerable element 30 of the present invention can be deployed to manoeuvre and/or expand the stent graft 82 into apposition. The steerable element 30 can also be integrated into the implant, for example as part of a delivery system, to impart a permanent curve on the stent graft 82 for improved conformity.

FIG. 13 illustrates a bespoke catheter system comprising a plurality of steerable elements 30A/30B/30C at different longitudinal positions on the catheter 70. In this particular example, three steerable element 30A/30B/30C are provided, and the system is deployed within the same anatomical context as the catheter 70 of FIGS. 10A and 10B. The advantages relative to the prior art include those discussed above with reference to FIGS. 10A and 10B. The additional steerable elements 30B/30C in the region of tortuous anatomy near the proximal end 72 help further to reduce unwanted forces and stress on delicate tissues. The positions and/or configurations of the steerable elements 30A/30B/30C (e.g. the way in which they will deform on inflation of their inflatable members and their relative orientations) can be determined by reference to measurements of the patient's anatomy, for example using imaging data. Each of the steerable elements 30A/30B/30C/30D may be actuatable independently of the others or in combination with the others. For example, each steerable element 30A/30B/30C/30D may have its own inflatable member, and a pressure control system may be provided that is capable of independently controlling each of the pressures in the four inflatable members. Alternatively, the four inflatable members may be linked together so as to inflate at the same time, for example with a single internal fluid pressure applied to all four inflatable members. The approach of measuring the anatomy of a patient and configuring the steerable element or elements accordingly can also be applied where the steerable element or elements are implemented as part of a medical implant, such as a stent graft.

FIG. 14 illustrates a further embodiment of a bespoke system, where a catheter 70 having steerable elements (not shown explicitly) is applied to help navigate through coronary blood vessels and achieve a resilient delivery system. As before, the region of application is the heart, with aortic arch 4, head and neck vessels 8/10/12/14 and vena cava 90 shown for reference. The steerable elements are positioned at points A, B, C, D, and E. Without these steerable elements, the catheter 70 would have to press harder against the walls in order to maintain the required curvature, which would risk irritation or damage to delicate tissue in these regions.

1. A steerable element for use in surgery, comprising:
an inflatable member; and
an elongate frame azimuthally surrounding said inflatable member, wherein:
said inflatable member is configured to press against said elongate frame on inflation so as to cause a change in the curvature of said elongate frame.

2. An element according to claim 1, wherein:
said change in said curvature is an increase in curvature.

3. An element according to claim 1, wherein:
said elongate frame has a first end and a second end, the first end being longitudinally spaced apart from the second end; and
said change in said curvature causes an increase in the angle between a longitudinal axis associated with said first end of said elongate frame and a longitudinal axis associated with said second end of said elongate frame.

4. An element according to claim 1, wherein:
said change in said curvature comprises a decrease in the average radius of curvature of the longitudinal axis of said elongate frame.

5. An element according to claim 1, wherein said elongate frame comprises:
a first spine, extending longitudinally, and displaced laterally away from the longitudinal axis of said elongate frame.

6. An element according to claim 5, wherein said first spine is longitudinally continuous.

7. An element according to claim 5, wherein said elongate frame further comprises:
a plurality of ring members surrounding the longitudinal axis of said elongate frame, and spaced apart from each other along the longitudinal axis, each of said plurality of ring members being connected to said first spine, wherein:
the element is configured such that, on inflation of said inflatable member said first spine constrains relative movement of said ring members with the result that the separation between adjacent ones of said ring members increases more on the side of the longitudinal axis of said elongate frame opposite to said first spine than at said first spine.

8. An element according to claim 7, wherein each of said plurality of rings is centered on said longitudinal axis of said elongate frame.

9. An element according to claim 5, wherein said elongate frame further comprises:
a second spine, extending longitudinally, and displaced laterally away from said longitudinal axis of said elongate frame.

10. An element according to claim 9, wherein, relative to said first spine, said second spine has at least one of the following: a different length, a different width, a different thickness, a different composition, a different spacing away from said longitudinal axis of said elongate frame.

11. An element according to claim 9, wherein said second spine extends over a different range of longitudinal positions, compared with said first spine.

12. An element according to claim 9, wherein said second spine is positioned at a different azimuthal angle compared with said first spine.

13. An element according to claim 9, wherein said first and second spines are configured such that inflation of said inflatable member causes bending of said elongate member about a first axis within a first range of longitudinal positions, and about a second axis within a second range of longitudinal positions, said first and second axes being non-parallel.

14. An element according to claim 9, wherein said first and second spines are configured such that inflation of said inflatable member causes bending of said elongate member about a first axis within a first range of longitudinal positions, and about a second axis within a second range of longitudinal positions, said first and second axes being parallel to each other.

15. An element according to claim 13, wherein said bending is in a single, first sense within said first range of positions and is in a single, second sense, opposite to said first sense, within said second range of positions.

16. An element according to claim 7, wherein said elongate frame is configured to resist inflation of said inflatable member to an extent that varies as a function of longitudinal position along said elongate frame.

17. An element according to claim 16, wherein the spacing between adjacent ones of said plurality of rings varies as a function of longitudinal position along said elongate frame.

18. An element according to claim 17, wherein the tensile strength of said plurality of rings varies as a function of longitudinal position along said elongate frame.

19. An element according to claim 1, wherein said elongate frame is configured to resist inflation of said inflatable member to an extent that varies as a function of longitudinal position along said elongate frame, said element being configured such that:
said inflatable member inflates progressively from one longitudinal end of the inflatable member towards the other longitudinal end, as the pressure inside said inflatable member is increased, so as to cause said elongate frame to bend progressively from said one longitudinal end towards said other longitudinal end.

20-26. (canceled)

27. An insertion system for a medical procedure, comprising:
a steerable element according to claim 1; and
a pressure control system configured to control the fluid pressure within the inflatable member, wherein said pressure control system is capable of selectively maintaining one of a continuous range of pressures in order selectively to impose one of a continuous range of possible curvatures of the elongate frame, or elongate frames, with which the inflatable member is coupled.

28-30. (canceled)

31. A method of configuring a steerable element for use in surgery, wherein:
said steerable element is according to claim 1; and
said method comprises:
obtaining data representing the morphology of a cavity within a human or animal body;
configuring the elongate frame such that inflation of said inflatable member will cause said elongate frame to adopt a shape that corresponds to the morphology of said cavity.

32-33. (canceled)
